(19) United States
(12) Patent Application Publication
(10) Pub. No.: US 2023/0347012 A1
(43) Pub. Date: Nov. 2, 2023

(54) BIORESORBABLE EMBOLIZATION PARTICLES

(71) Applicant: Regents of the University of Minnesota, Minneapolis, MN (US)

(72) Inventors: Chen Guo, Edina, MN (US); Jafar Golzarian, Plymouth, MN (US)

(21) Appl. No.: 18/042,573

(22) PCT Filed: Aug. 31, 2021
(86) PCT No.: PCT/US2021/071325

§ 371 Date: Feb. 22, 2023

Related U.S. Application Data
(60) Provisional application No. 63/072,464, filed on Aug. 31, 2020.

(51) Int. Cl. A61L 24/10; C08F 20/56; A61L 24/00

(52) U.S. Cl. CPC A61L 24/108; C08F 20/56; A61L 24/0015; A61L 2430/36

(57) ABSTRACT

The present disclosure is generally directed to an embolic material in the form of a microparticle. The embolic material generally includes an alkene functionalized biopolymer. For instance, the embolic material may include a methacrylamide functionalized biopolymer. In some examples, the embolic material may further comprise a therapeutic agent, such as doxorubicin.

This application claims priority to U.S. Provisional Patent Application 63/072,464, filed Aug. 31, 2020, the entire content of which is incorporated by reference.

### TECHNICAL FIELD

The disclosure relates to embolic materials.

### BACKGROUND

Transcatheter arterial embolization (TAE) has been widely accepted for its efficacy in treating various diseases including tumors, vascular lesions, and hemorrhages. For a safe and effective treatment, the selection of an appropriate embolic material is important.

### SUMMARY

In one aspect, the disclosure is directed to an embolic material including a particle comprising an alkene functionalized biopolymer.

In another aspect, the disclosure is directed to an embolization suspension comprising a solvent and a plurality of embolic particles suspended in the solvent. The embolic particles include an alkene functionalized biopolymer.

In a further aspect, the disclosure is directed to a kit comprising a plurality of embolic particles and a syringe or vial in which the plurality of embolic particles is disposed. The plurality of embolic particles include an alkene functionalized biopolymer.

In an additional aspect, the disclosure is directed to a method of forming an embolic material. The method includes introducing a functional group containing a carbon-carbon double bond on a biopolymer to form an alkene functionalized biopolymer; and crosslinking the alkene functionalized biopolymer to form a plurality of embolic particles.

In a further aspect, the disclosure is directed to a method comprising injecting an embolic particle comprising an alkene functionalized biopolymer in a blood vessel of a patient to occlude an artery of the patient.

The details of one or more embodiments are set forth in the accompanying drawings and the description below. Other features, objects, and advantages of the disclosure will be apparent from the description and drawings, and from the claims.

### BRIEF DESCRIPTION OF DRAWINGS

FIG. 1 is a flow diagram of an example technique for producing embolic particles including an alkene functionalized biopolymer.

FIG. 2 is an image of gel electrophoresis results comparing methacrylamide functionalized whey protein, unmodified whey protein, and a comparative molecule.

FIG. 3 is a plot comparing methacrylamide functionalized whey protein and unmodified whey protein.

FIG. 4 shows a micrograph of methacrylamide functionalized whey protein aqueous solution droplets suspended in mineral oil before reaction.

FIG. 5 is a plot of the size distribution of the synthesized microspheres.

FIG. 6 is a plot of doxorubicin content versus time when loading embolic microspheres formed from methacrylamide functionalized whey protein with doxorubicin.

FIG. 7 is a plot of doxorubicin content versus time when loading embolic microspheres formed from methacrylamide functionalized whey protein with doxorubicin.

FIG. 8 is a plot of doxorubicin content versus time during doxorubicin release from embolic microspheres formed from methacrylamide functionalized whey protein.

FIG. 9 is a plot of doxorubicin content versus time during doxorubicin release from embolic microspheres formed from methacrylamide functionalized whey protein.

FIG. 10 is a plot of microsphere degradation over time for microspheres formed from methacrylamide functionalized whey protein.

FIG. 11-22 are micrographs of examples of microspheres formed from methacrylamide functionalized whey protein after introduction through a catheter.

FIG. 23 is a photograph of microspheres formed from methacrylamide functionalized whey protein suspended in a mixture of saline and contrast media.

FIG. 24 is a plot of example loading of doxorubicin on microspheres formed from methacrylamide functionalized whey.

FIGS. 25-28 are micrographs of examples of microspheres formed from methacrylamide functionalized whey protein after drying and rehydration.

### DETAILED DESCRIPTION

The present disclosure is generally directed to an embolic material which, in some examples, may be in the form of a particle or a plurality of particles (e.g., microspheres, microcylinders, or the like). The embolic material generally includes an alkene functionalized biopolymer, such as a methacrylamide functionalized protein.

Temporary embolization may be accomplished by a material that is biocompatible, bioresorbable, and compressible. However, these properties are not easily achieved in a single embolic particle. For example, crosslinking of polymers may be accomplished by using a small molecule crosslinking agent, such as glutaraldehyde. While use of the small molecule crosslinking agent facilitates the desired crosslinking reaction, if the crosslinked polymer is biodegradable and degrades in a body of a patient, some small-molecule crosslinking agents may be toxic or have other adverse effects on cells or tissue in the body of the patient.

In general, the disclosure is directed to an embolic material comprising a polymerizable alkene functionalized biopolymer (e.g., a methacrylamide functionalized protein). The biopolymer may include at least one amine functional group. The alkene functionalized biopolymer may be prepared by introducing an amide group through reacting the amine functional group. Example biopolymers include soy protein, whey protein, casein protein, pea protein, egg protein, quinoa protein, collagen, gelatin, or the like. The amide functional group may enable crosslinking of the alkene functionalized biopolymer alone, or with a second molecule or polymer (either synthetic or biopolymer). By using a biopolymer, the embolic material may exhibit biocompatibility, biodegradability, and be safe for human in vivo use. Further, using alkene functionalized biopolymers may allow crosslinking without use of a small-molecule crosslinking agent. Some small-molecule crosslinking agents may be toxic, so avoiding use of one may improve biocompatibility of the resulting embolic material.

In some examples, the alkene functionalized biopolymer may be crosslinked using a second molecule. For example, the second molecule may include a functional group that reacts with a carbon-carbon double bond. In some examples, the second molecule may include another alkene containing monomer, such as a multifunctional polyethylene glycol (PEG) acrylate (e.g., a difunctional PEG methacrylate) or a thiol containing monomer, such as a multifunctional PEG thiol or thiol containing proteins such as keratins. The second molecule may affect properties of the embolic material, such as water solubility, fracture toughness, drug loading capacity, drug release time, degradation or the like.

The alkene functionalized biopolymer may be polymerized using any free radical polymerization scheme, including thermally initiated free radical polymerization, photoinitiated free radical polymerization, or the like. In some examples, the polymerization may be conducted in an oil-water emulsion, which may result in substantially spherical embolic particles. In other examples, the polymerization may be conducted using a photomask, which may enable generation of embolic particles with different shapes, such as cylinders, cubes, prisms, or the like.

Due to the use of biopolymers, the embolic materials described herein are expected to be biodegradable and biocompatible. Additionally, because the embolic material comprises a three-dimensional network of crosslinked biopolymers, the mechanical properties, such as, for example, the compressibility of the embolic particles, may be sufficient to permit introduction of the embolic particles into an artery of a patient through a syringe, catheter, or the like.

In some examples, the embolic materials may be used to introduce a drug or other therapeutic agent to a selected site within a patient. For instance, the embolic material may be used as a carrier for a therapeutic agent, such as an anti-cancer agent. One example of a therapeutic agent which may be loaded into the embolic particles is doxorubicin.

In some examples, the embolic materials include an alkene functionalized whey protein. An alkene functionalized whey protein may react relatively quickly (e.g., on the scale of a few minutes) and the reaction may be biocompatible. This may allow relatively inexpensive manufacture of embolic particles from alkene functionalized whey protein, and may allow encapsulation of biological materials, such as cells, within alkene functionalized whey protein particles. Further, in some implementations, the reaction may be photo-initiated, and may use one of a variety of wavelengths. Some of the wavelengths may be produced by relatively inexpensive light emitting diodes (LEDs), such as commercially available LED lights. This may further contribute to low cost of the embolic particles.

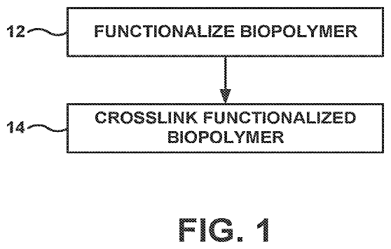

FIG. 1 is a flow diagram illustrating an example technique for forming an embolic material including an alkene functionalized biopolymer. The biopolymer may include any naturally occurring biopolymer that includes amine functionality to which an alkene group may be attached. For example, the biopolymer may include a protein that includes lysine, asparagine, or arginine amino acids, which include side chain amine groups. Some example bioproteins that may be used include whey proteins (e.g., α-lactalbumin, β-lactoglobulin, and/or bovine serum albumin), soy proteins, casein proteins, pea proteins, egg proteins, quinoa proteins, collagen, gelatin, or the like.

Initially, a biopolymer may be alkene functionalized through reacting a molecule including an alkene functional group with an amine group on the biopolymer (12). One reaction that introduce a methacrylamide group on a biopolymer is illustrated in Reaction 1:

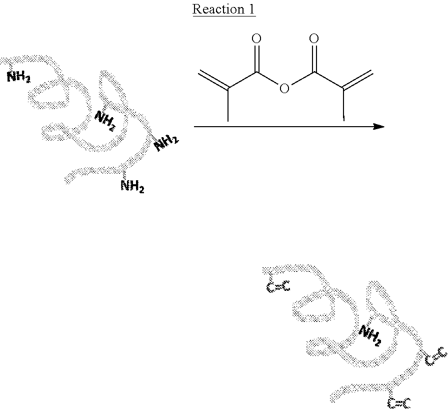

In Reaction 1, the amine groups on a single biopolymer are reacted with a methacrylic anhydride to provide a carbon-carbon double bond functional group. Although a methacrylic anhydride is shown in Reaction 1, any suitable molecule such as acrylic acid, methacrylic acid, acrylic anhydride and so on that can introduce an alkene functional group through the reaction with an amine group may be used to modify the biopolymer to be polymerizable. As shown in Reaction 1, at least some amine groups within the biopolymer may react with the methacrylic anhydride molecule. For example, some amine groups may not react, and may still include amine functionality after Reaction 1 is performed. Other amine groups may be modified with methacrylamide groups. The biopolymer may include a weight average molecular weight of between about 10,000 daltons (Da; equivalent to grams per mole (g/mol)) and about 100,000 Da. In some embodiments, a weight average molecular weight of the biopolymer may be about 20,000 Da.

The degree of alkene (e.g., methacrylamide) functionalization of the biopolymer may be affected by, for example, the molar ratio of the molecule that includes the alkene functionality to amine-containing units (e.g., amino acids that include a side chain with an amine group, such as lysine, asparagine, or arginine). An increased molar ratio of the molecule that includes the alkene functionality to amine-containing units may result in greater alkene functionalization of the biopolymer, which in turn may lead to greater crosslinking density when the alkene containing biopolymer is reacted to form the embolic particles. Conversely, a decreased molar ratio of the molecule that includes the alkene functionality to amine-containing units may result in lesser alkene functionalization of the biopolymer, which in turn may lead to lower crosslinking density when alkene-containing biopolymer is reacted to form the embolic particles. In some examples, the crosslinking density may be approximately proportional to the degree of alkene functionalization of the biopolymer. In some embodiments, a greater crosslinking density may lead to greater mechanical strength (e.g., fracture strain). During alkene functionalization of a biopolymer, multiple biopolymer molecules are alkene functionalized, and each biopolymer molecule may exhibit a respective degree of modification of the amine groups into alkene containing groups.

Once the biopolymer has been modified, the alkene functionalized biopolymer may be crosslinked (14). The alkene functionalized biopolymer may be dissolved in a solvent, such as water. For example, about 200 milligram (mg) of alkene functionalized biopolymer may be mixed in about 1 milliliter (mL) of water to form a 20% weight/volume (w/v) solution. Of course, solvents other than water may be used, and solutions having other concentrations of alkene functionalized biopolymer may be used. For example, saline or phosphate-buffered saline (PBS) may be utilized as alternative solvents. The solution may have a concentration of alkene functionalized biopolymer between about 5 w/v % (e.g., g/mL) and about 25 w/v %.

In some examples, the alkene functionalized biopolymer may be crosslinked alone (e.g., to itself, without another type of molecule). The alkene functionalized biopolymer may be polymerized using any suitable free-radical initiated polymerization reaction. Any suitable free radical initiator may be used to initiate the polymerization. Suitable free radical initiators include those that generate free radicals through thermal decomposition, photolysis, redox reactions, dissociation of persulfates, ionizing radiation, electrolysis, or the like. For example, the free radical initiator may be a photoinitiator, such as lithium phenyl-2,4,6-trimethylbenzoylphosphinate (LAP), 2-hydroxy-4′-(2-hydroxyethoxy)-2-methylpropiophenone (IRGACURE 2959), benzophenone, phenyl bis (2,4,6-trimethylbenzoyl) phosphine oxide (BAPO, IRGACURE 819), 2-hydroxy-2-methyl-1-phenyl-propan-1-one (IRGACURE 1173), 2,2′-azobis[2-methyl-n-(2-hydroxyethyl) propionamide (VA-086), 2,2-dimethoxy-2-phenylacetophenone (IRGACURE 651 or DMPA), diphenyl(2,4,6-trimethylbenzoyl)phosphine oxide (Darocure TPO; Lucirin TPO),ethyl (2,4,6-trimethylbenzoyl)phenylphosphinate, or the like. Free radical polymerization, such as photopolymerization, may provide control of particle size and particle size distribution of the resulting embolic material, e.g., by allowing uniform mixing of an oil-water emulsion in which the reaction is performed or by allowing use of a photomask.

In other examples, the alkene functionalized biopolymer may be reacted (e.g., crosslinked) with another molecule. For example, the alkene functionalized biopolymer may react with a second molecule, such as a second polymer, that includes one or more functional groups that react with the carbon-carbon double bond present in the alkene group of the alkene containing biopolymer. The functional group may include, for example, a thiol, an amine, or the like. Example second molecules may include a difunctional polyethylene glycol (PEG) acrylate. The second molecule may be used to affect properties of the resulting embolic material. For instance, the second molecule may be used to affect fracture toughness, hydrophilicity or hydrophobicity, drug loading, drug release, degradation or the like. For instance, the second molecule may be used to increase fracture toughness of the embolic material, which may facilitate introduction of the embolic material through a catheter, needle, or the like. As another example, the second molecule may be used to increase hydrophilicity of the embolic material, which may facilitate suspension of the embolic material in an aqueous medium, such as water, saline, or the like.

In some examples, the crosslinking reaction may be conducted in a water-oil emulsion. The reactants may be dissolved in water and mixed with an oil to form the emulsion. The resulting embolic material may include microspheres.

In some implementations, the oil may include mineral oil. In other examples, the oil may include food grade oil, such as canola oil. This may contribute to benignity of the reaction mixture, may contribute to low cost of the embolic particles, or both. In some implementations, the reaction may be a “green” reaction in that it uses environmentally benign chemicals. Further, in some instances, the oil may be recovered after the reaction and reused or recycled.

In other examples, the crosslinking reaction may be conducted using a photomask to control the shape and size of the embolic material. The photomask system may include two layers. A top layer may be formed from a material that is substantially opaque to the wavelength of light used in the polymerization process. The top layer of material may define a plurality of apertures through which light can pass. The polymerization reactants may be contained between the top layer of material and a bottom layer of material. The photomask system also may include a spacer between the bottom layer and top layer. The spacer spaces the bottom layer and the top layer and defines the thickness of the polymerization reactants. By selecting the shape of the apertures and the size of the spacer, the shape of the embolic material particles may be defined. For instance, by using a photomask with circular apertures and a spacing between the bottom and top layer that is larger than a diameter of the circular apertures, substantially cylindrical embolic particles may be formed.

In some examples, the reaction may be performed in a microfluidic device. The microfluidic device may be used to control mixing of reactants, exposure to reaction initiators, reaction dwell time, or the like, which may facilitate control of average particle size and particle size distribution.

Regardless of the reaction scheme (e.g., using a photomask or an oil-water emulsion), an extent of crosslinking may affect mechanical properties of the resulting embolic particle. For example, a greater crosslinking density generally may provide greater mechanical strength (e.g., fracture strain), while a lower crosslinking density may provide lower mechanical strength (e.g., fracture strain). The crosslinking density may also affect the degradation rate of the embolic particles. For example, a greater crosslinking density may lead to a longer degradation time, while a lower crosslinking density may lead to a shorter degradation time. In some examples, the crosslink bonds may degrade through enzyme degradation along the protein molecule chain. The degree of crosslinking also impacts the swelling of the embolic particles, where the higher-crosslinked may swell to sizes smaller than the lower-crosslinked embolic particles.

In some embodiments, the reaction conditions may be selected to result in particles with a mean or median particle size between about 50 μm and about 2200 μm. In some examples, the reaction conditions may be selected to result in particles with a mean or median particle size of less than about 2000 μm, particles with a mean or median particle size of between about 100 μm and about 1200 μm, particles with a mean or median particle size of between about 100 μm and about 300 μm, particles with a mean or median particle size of between about 300 μm and about 500 μm, particles with a mean or median particle size of between about 500 μm and about 700 μm, particles with a mean or median particle size of between about 700 μm and about 900 μm, particles with a mean or median particle size of between about 900 μm and about 1200 μm, or particles with a mean or median particle size of between about 1600 μm and about 2000 μm.

In some examples, particles with different mean or median diameters may be used for different applications. For example, in some implementations, particles with a mean or median particle size between about 100 μm and about 300 μm may be loaded with a therapeutic agent, such as a chemotherapeutic agent as described further below, and used to deliver the therapeutic agent to a therapy site, while also embolizing blood vessels with a diameter similar to the mean or median particle size of the particles. In some examples, particles with a mean or median particle size between about 300 μm and about 500 μm may be used similarly, and loaded with a therapeutic agent. In some examples, particles with a larger mean or median diameter may be used as embolization materials, and may not be loaded with a therapeutic agent.

The embolic particles may be packaged for distribution in various ways. For example, the embolic particles may be distributed as part of a kit. In some embodiments, the kit may include the embolic particles disposed in a syringe or a vial. The kit may optionally include a catheter, a guide wire, and/or a container of solution in which the embolic particles are to be suspended. The catheter may be used to inject the embolic particles into a blood vessel of a patient. The guide wire may be used to position the catheter within the blood vessel.

In some examples, the kit may be an emergency trauma kit for acute embolization in massive bleeding trauma. Such a kit may include, for example, a syringe or vial and a plurality of embolic particles disposed in the syringe or vial. In some examples, the embolic particles may comprise an average diameter of between about 1600 μm and about 2000 μm. In other examples, the embolic particles may comprise a different average diameter, such as an average diameter within a range listed in other portions of this application. In some examples, the kit may further include a catheter, a guide wire for positioning the catheter within a blood vessel, such as an artery, of the patient, and/or a container of solution in which the embolic particles are to be suspended. Prior to injection of the embolic particles, the solution may be aspirated into the syringe to form a suspension of the embolic particles in the solution.

The embolic particles may be used to embolize arteries to treat various conditions, including, for example, an arteriovenous malformation, a cerebral aneurysm, gastrointestinal bleeding, an epistaxis, primary post-partum hemorrhage, or the like.

In some examples, in addition to being utilized as an embolizing agent, the embolic particles may be used to deliver a therapeutic agent to a therapy site. The embolic particles may carry therapeutic agent due to functional groups on alkene functionalized biopolymers. For example, the embolic particles may be loaded with a therapeutic agent, such as a chemotherapeutic agent, and used to deliver the chemotherapeutic agent to a tumor and/or to embolize arteries that feed the tumor. In other examples, the embolic particles may be loaded with a cell, a bioactive molecule, or another drug.

An example of a therapeutic agent that may be loaded into the embolic particles is doxorubicin (available under the trade designation Adriamycin from Selleck Chemicals LLC, Houston, Texas, U.S.A.). Doxorubicin includes a protonated amino group and a plurality of hydroxyl groups, which may interact with functional groups, such as a carboxylic group, in the embolic particles to bind to the embolic particles via ionic interactions. While doxorubicin is provided as one example of a therapeutic agent which may be loaded into the embolic particles of the present disclosure, other therapeutic agents may be used with the embolic particles. For example, hydrophilic therapeutic agents may be utilized with the embolic particles according to the disclosure. In particular, therapeutic agents that include at least one functional group that interacts with a carboxylic group, hydroxyl group or an aldehyde group are expected to be compatible with embolic particles of the present disclosure. Examples of such therapeutic agents include irinotecan (available under the trade designation Camptosar® from Pfizer, New York, New York, U.S.A), ambroxol, and other therapeutic agents with at least one positively charged functional group. In some examples, in addition to ionic interactions between the therapeutic agent and the alkene functionalized biopolymer, the therapeutic agent may adsorb or adsorb in the embolic particles.

In some examples, the therapeutic agent may be loaded into the embolic particles during formation of the embolic particles, i.e., during the crosslinking of alkene functionalized biopolymer. In such examples, the therapeutic agent may be deposited into the emulsion or reaction mixture along with the alkene functionalized biopolymer and any other reactants. As the embolic particles form, the therapeutic agent may load into the embolic particles.

In other examples, the therapeutic agent may be loaded into the embolic particles after formation of the embolic particles. For example, the embolic particles may be immersed in a solution of the therapeutic agent in a solvent, such as saline or a saline and contrast medium mixture, to load the therapeutic agent into the embolic particles. In some examples, the therapeutic agent solution may have a concentration of between about 1 mg therapeutic agent per mL solvent (mg/mL) and about 2 mg/mL.

In some examples, the therapeutic agent may be loaded into the embolic particles to a concentration of between about 0.3 mg therapeutic agent per mg dry embolic particle (mg/mg) and about 0.75 mg/mg.

Embolic particles formed according to the present disclosure may be utilized for a number of applications. For example, one application for an embolic particle including alkene functionalized biopolymer is transarterial chemoembolization (TACE) of liver tumors. TACE for unresectable hepatocellular carcinoma (HCC) is an approved treatment modality that increases patient survival compared to intravenous chemotherapy. TACE includes intraarterial (via the hepatic artery) injection of chemotherapeutic agents followed by embolization of tumoral feeding arteries. The trend in TACE is to use drug eluting beads loaded with chemotherapeutic agents that are progressively released into the tumor. Drug eluting TACE is associated with less systemic toxicity and a better patient tolerance. Because the embolic particles including alkene functionalized biopolymers is bioresorbable and is thus absorbed by the body of the patient over time after injection, the release profile of the chemotherapeutic agents may be controlled. Additionally, the embolic particles including alkene functionalized biopolymers may act as combination chemotherapeutic agent carriers and embolization agents. Furthermore, because the embolic particles including alkene functionalized biopolymers are bioresorbable, artery integrity may be restored upon resorption, which may be advantageous in some examples.

Another application for embolic particles including alkene functionalized biopolymers is Uterine Fibroids Embolization (UFE). Uterine Fibroids are benign muscular tumors that grow in the wall of the uterus. Uterine fibroids can grow as a single tumor or as many tumors. Uterine fibroids can be either as small as an apple seed or as big as a grapefruit. In unusual cases uterine fibroids can become very large. An increasingly accepted therapy technique for uterine fibroids is UFE. The main purpose of UFE is to reduce the size of the fibroid and to treat excessive uterine bleeding. In essence, UFE involves the placement of a catheter into the uterine arteries and injection of embolic particles into the uterine arteries to achieve fibroid devascularzation and progressive shrinkage. Use of bioresorbable embolic particles including alkene functionalized biopolymers may facilitate restoration of uterine artery integrity after embolization.

### EXAMPLES

### Example 1: Preparation of Methacrylamide Functionalized Whey Protein

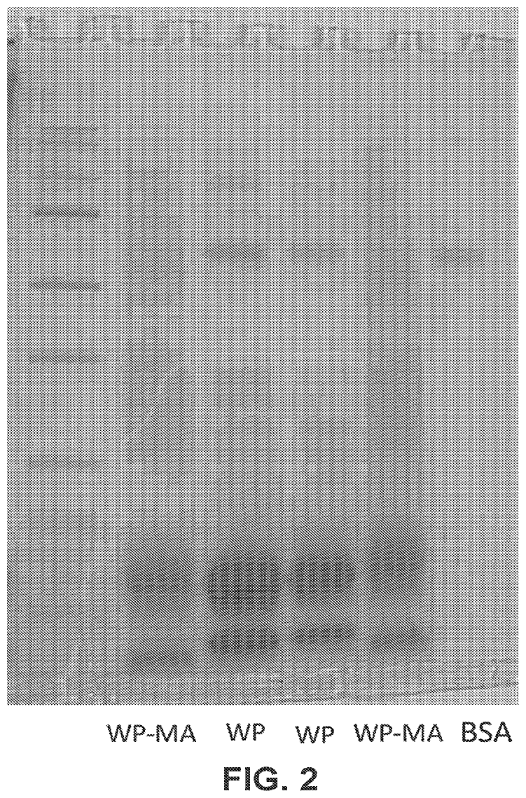

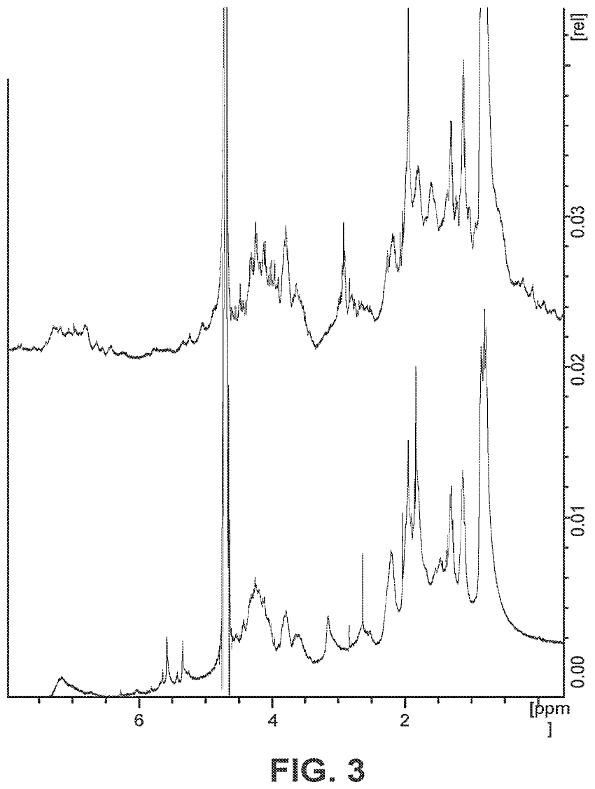

About 2.5 g of whey protein (available from Bulk Apothecary, Aurora, Ohio), M_{w} about 20,000 g/mol) and 15 mL distilled water were added to a 250 mL flask. After the whey protein dissolved substantially completely, 450 mg methacrylic anhydride (about two times molar equivalent of lysine content in whey protein) was added to the flask. The pH of the reaction solution was adjusted to about 8 and the reaction was allowed to proceed for 18 to 24 hours at about 23° C. The pH of the reaction solution was then adjusted to 7 and dialyzed against water using a regenerated cellulose dialysis tube (MWCO 6000-8000 Da). As shown in FIGS. 2 and 3, the methacrylamide functionalization had no significant effect on the whey protein molecular weight. The modified whey protein had about 70% conversion of amine groups into methacrylamide groups.

### Example 2: Crosslinking of Methacrylamide Functionalized Whey Protein

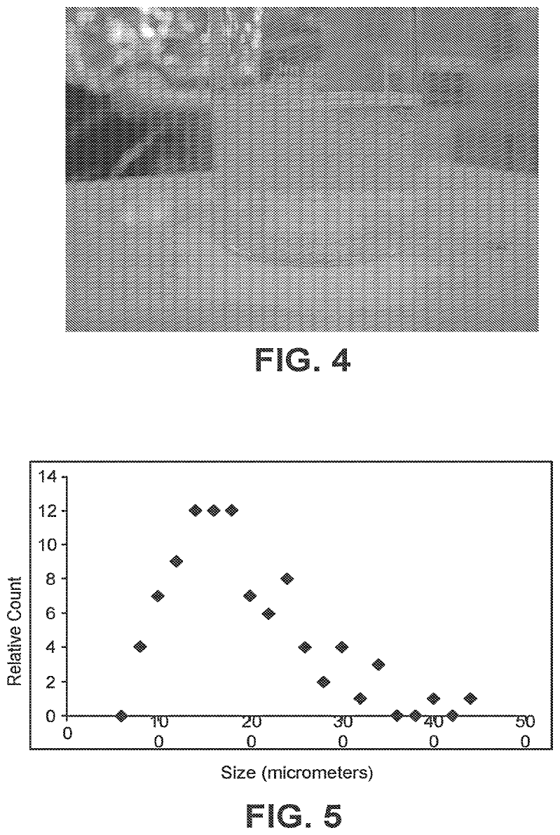

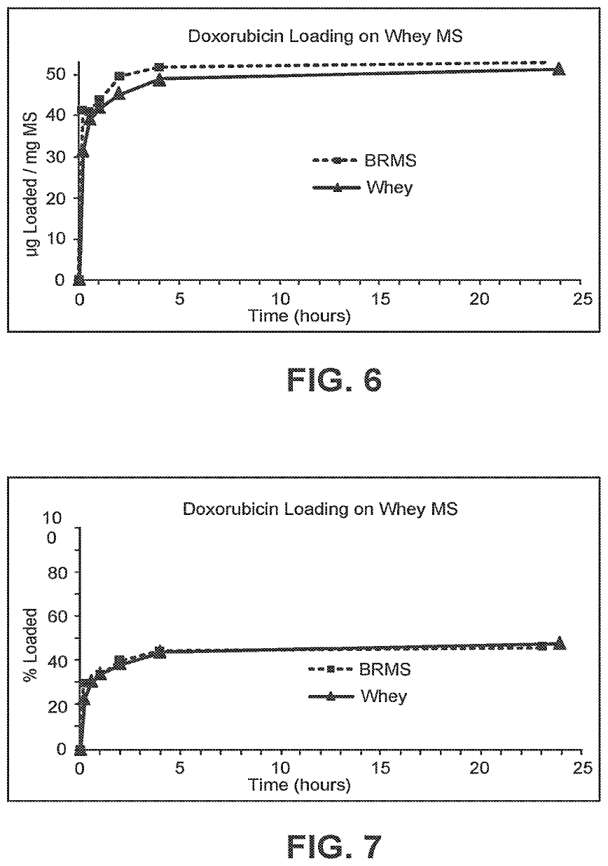

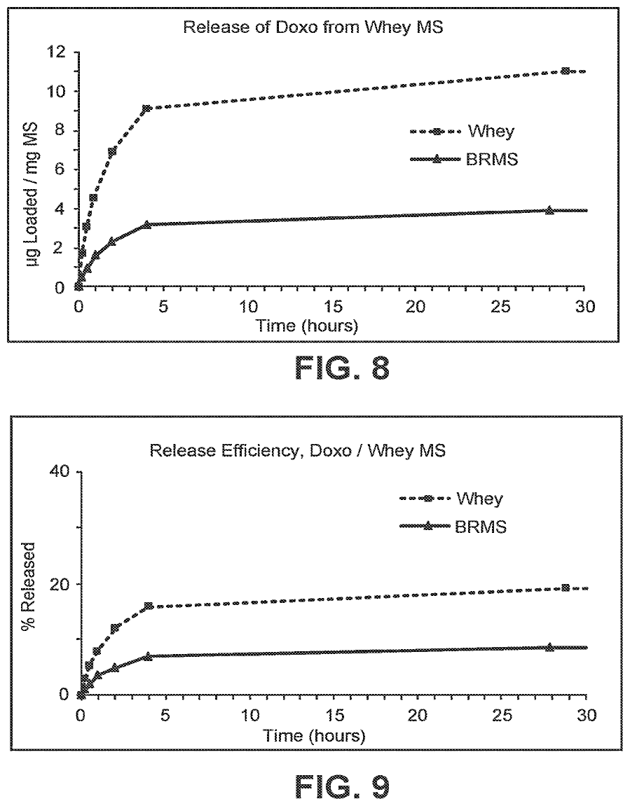

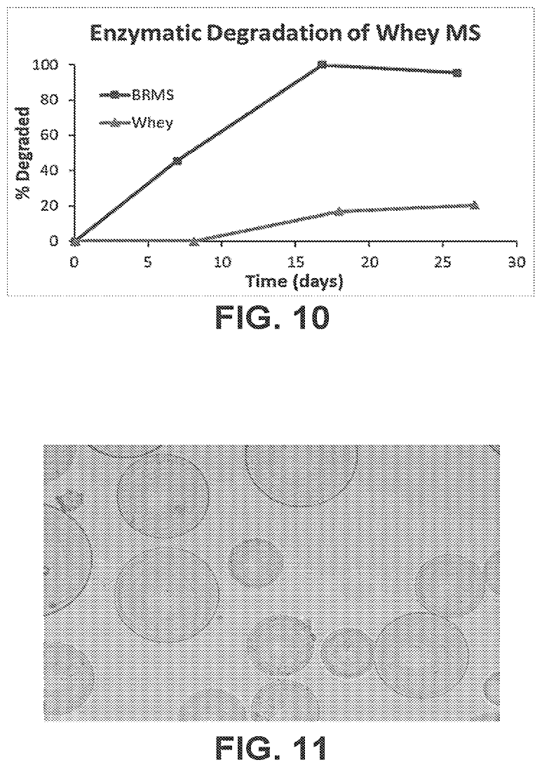

58.8 mg of lithium phenyl-2,4,6-trimethylbenzoylphosphinate (LAP) was added into 10 mL distilled water to make a 20 mM LAP solution. About 0.5 g of methacrylamide functionalized whey protein prepared by the reaction described in Example 1 was placed in 2 mL of 20 mM LAP solution in a flask. A 1.5 mL aliquot of this solution was added to 15 mL of light mineral oil containing 45 μL of Span® 80 (a sorbitane monooleate available from Millipore Sigma, Burlington, Massachusetts), while stirring at 250 RPM. A beam of UV light at 405 nm generated by a collimated LED (available from Thorlabs, Inc., Newton, New Jersey) at light intensity of 165 mW/cm², and the emulsion was illuminated from above for about 10 minutes. The resulting polymerized microspheres were washed in 5% TWEEN®/saline (TWEEN® is available from Millipore Sigma, Burlington, Massachusetts) and rinsed in saline. The microspheres had a size range of approximately 60-450 μm. FIG. 4 shows a micrograph of methacrylamide functionalized whey protein aqueous solution droplets suspended in mineral oil before reaction. FIG. 5 is a plot of the size distribution of the synthesized microspheres.

### Example 3: Drug Loading and Releasing

Loading of the microspheres with doxorubicin was performed by immersing 60 mg (wet weight) of microspheres prepared according to Example 2 in 4 mL of a 2 mg/mL doxorubicin hydrochloride/water solution, with gentle agitation at room temperature. At various time points six 255 μL samples of the doxorubicin hydrochloride/water solution were taken, excluding any microspheres. The samples were analyzed for doxorubicin concentration by measuring optical absorption at 483 nm and compared to a standard curve. The concentrations of doxorubicin were then determined by a subtractive calculation, taking into account the volumes removed. The calculated amount of drug eliminated from the loading solution was taken as the amount loaded into the microspheres, and plotted against time.

The release experiment was initiated by first loading doxorubicin onto the microspheres. Approximately 46 μg of wet microspheres were immersed in 3.067 μL of a 2 mg/mL solution of doxorubicin hydrochloride for two hours at room temperature. Release was performed by immersing the loaded microspheres in approximately 3.067 μL of phosphate buffered saline release medium, and incubating with gentle agitation at 37° C. Six samples, each consisting of 50% of the release volume (approximately 1.534 μL), were withdrawn at various time points, excluding any microspheres, and replaced with an equal volume of fresh release medium which had been pre-warmed to 37° C. The samples were analyzed for doxorubicin concentration by measuring absorption at 483 nm and compared to a standard curve. The concentrations of doxorubicin were then determined by a subtractive calculation taking into account the volumes replaced. The calculated amount of drug added into the release solution was taken as the amount released from the microspheres, and plotted against time.

### Example 4: Degradation

The degradation experiment was performed by immersing approximately 80 mg of microspheres in 4 mL of a 10 μg/mL lysozyme/PBS degradation medium. Four replicate vials were set up in this way for each type of microsphere, one replicate for each time point, and incubated at 37° C. with gentle shaking for 27 days. At starting time and at each time point, one sample vial's contents were rinsed 3× with water, letting the remaining microspheres and fragments settle between each rinse, and stored at −20° C. Three times per week 80% of the degradation medium was exchanged (approximately 3.2 mL), excluding any microspheres or fragments. The samples were lyophilized, weighed, and the weight of remaining microspheres and fragments calculated. The percent degradation was calculated by comparing to the initial time point weights and plotted against time.

### Example 5: Catheter Delivery Testing

Microspheres as described above with Examples 1 and 2 with the following modifications. For a first set of samples, referred to as 16 wt. % microspheres, a solution with a ratio of 16 mg methacrylamide functionalized whey protein per 100 mg total solution was prepared with 20 mM LAP, methacrylamide functionalized whey protein, and water. For a second set of samples, referred to as 20 wt. % microspheres, a solution with a ratio of 20 mg methacrylamide functionalized whey protein per 100 mg total solution was prepared with 20 mM LAP, methacrylamide functionalized whey protein, and water. The 20 wt. % microspheres had a higher crosslink density, thus lower swelling, higher mechanical properties, and slower degradation.

A Boston Scientific Direxion Hi-FLO Fathom™ 16 catheter available from Boston Scientific, Marlborough, Massachusetts was filled with saline. The Boston Scientific Direxion Hi-FLO Fathom™ 16 catheter has an inner diameter of 0.027 inch (685.8 micrometers). About 0.2 mL (settled) 16 wt. % microspheres with a diameter between 500 micrometers and 710 micrometers in between 0.5 mL and 1.0 mL saline was placed in a 3 mL syringe and introduced through the catheter to a dish containing saline on a microscope stage. The microspheres were formed from methacrylamide functionalized whey protein. FIG. 11 is a micrograph of some of the microspheres after introduction through the Boston Scientific Direxion Hi-FLO Fathom™ 16 catheter. Microsphere compression was up to 3.4%, and the microspheres had no visible damage.

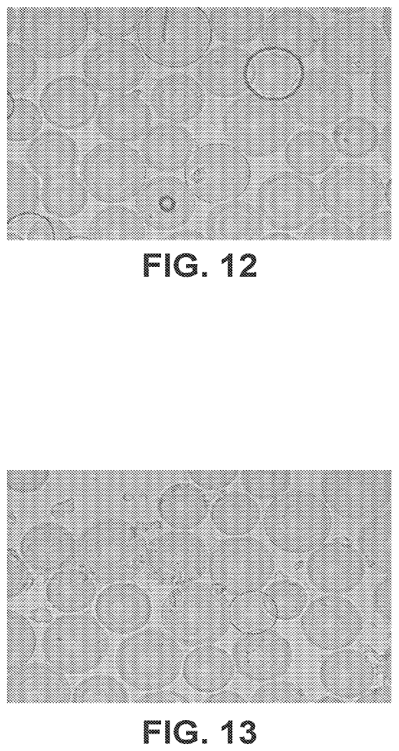

A Concentric Medical Trevo catheter available from Stryker Neurovascular, Fremont, California was filled with saline. The Concentric Medical Trevo catheter has an inner diameter of 0.017 inch (431.8 micrometers). About 0.2 mL (settled) 16 wt. % microspheres with a diameter between 300 micrometers and 500 micrometers (a maximum diameter of about 610 micrometers) in between 0.5 mL and 1.0 mL saline was placed in a 3 mL syringe and introduced through the catheter to a dish containing saline on a microscope stage. The microspheres were formed from methacrylamide functionalized whey protein. FIGS. 12 and 13 are micrographs of some of the microspheres after introduction through the Concentric Medical Trevo catheter. Microsphere compression was up to 13.7%, and the microspheres had minimal visible damage (equal to a volume of about 1 or 2 microspheres).

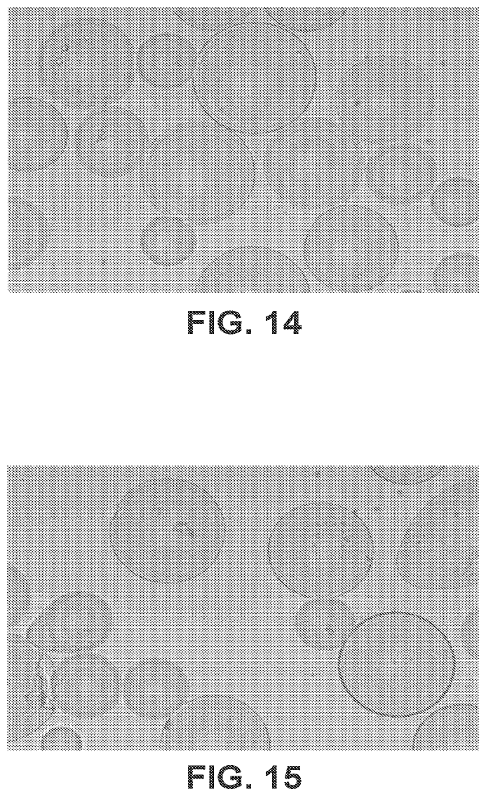

A Boston Scientific Fathom™ 16 catheter available from Boston Scientific, Marlborough, Massachusetts was filled with saline. The Boston Scientific Fathom™ 16 catheter has an inner diameter of 0.021 inch (533.4 micrometers). About 0.2 mL (settled) 16 wt. % microspheres with a diameter between 500 micrometers and 710 micrometers (a maximum diameter of about 786 micrometers) in between 0.5 mL and 1.0 mL saline was placed in a 3 mL syringe and introduced through the catheter to a dish containing saline on a microscope stage. The microspheres were formed from methacrylamide functionalized whey protein. FIGS. 14 and 15 are micrographs of some of the microspheres after introduction through the Boston Scientific Fathom™ 16 catheter. Microsphere compression was up to 24.7%, and, of the visualized microspheres, about eight larger microspheres had mild chipping or fracturing.

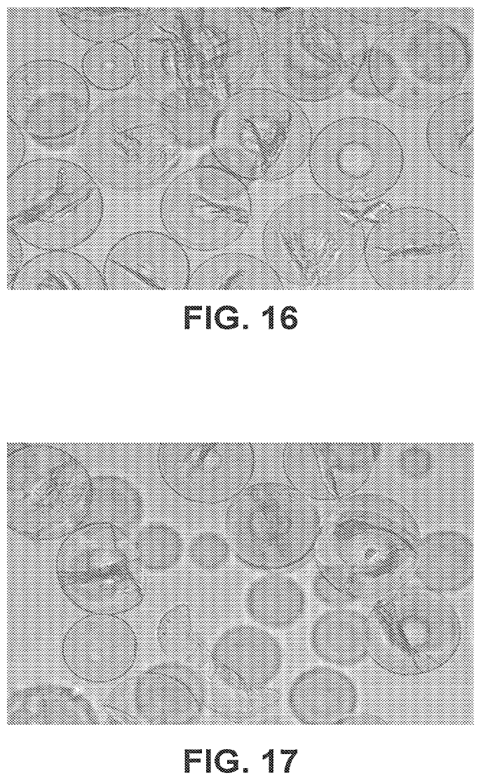

A Stryker Neurovascular Excelsior SL-10 catheter available from Stryker Neurovascular, Fremont, California was filled with saline. The Stryker Neurovascular Excelsior SL-10 catheter has an inner diameter of 0.0165 inch (419.1 micrometers). About 0.2 mL (settled) 16 wt. % microspheres with a diameter between 500 micrometers and 710 micrometers (a maximum diameter of about 707 micrometers) in between 0.5 mL and 1.0 mL saline was placed in a 3 mL syringe and introduced through the catheter to a dish containing saline on a microscope stage. The microspheres were formed from methacrylamide functionalized whey protein. FIGS. 16 and 17 are micrographs of some of the microspheres after introduction through the Stryker Neurovascular Excelsior SL-10 catheter. Microsphere compression was up to 40.9%, and the catheter clogged immediately. The catheter was cut in about 30 cm increments (to 96 cm) until the clog was cleared and the microspheres flowed through the catheter. Between about 50% and about 60% of the microspheres were damaged. The damaged microspheres generally had larger diameters. Of the damaged microspheres, most fractured at the surface or broke into large pieces.

From these examples, the fracture limit is between about 24.7% and about 40.9%.

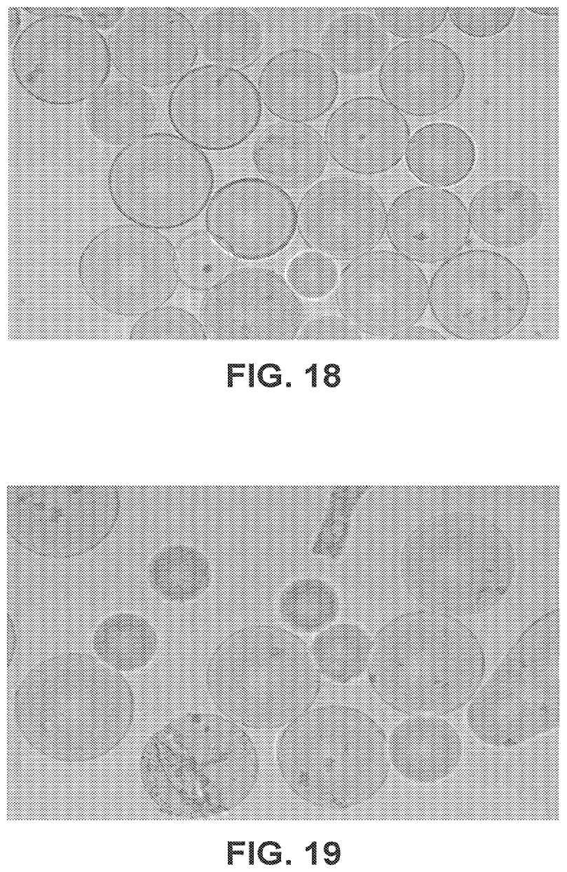

A Concentric Medical Trevo catheter was filled with saline. About 0.2 mL (settled) 20 wt. % microspheres with a diameter between 300 micrometers and 500 micrometers in between 0.5 mL and 1.0 mL saline was placed in a 3 mL syringe and introduced through the catheter to a dish containing saline on a microscope stage. The microspheres were formed from methacrylamide functionalized whey protein. FIG. 18 is a micrograph of some of the microspheres after introduction through the Concentric Medical Trevo catheter. Microsphere compression was up to 13.7%, and the microspheres had no visible damage.

A Boston Scientific Fathom™ 16 catheter was filled with saline. About 0.2 mL (settled) 20 wt. % microspheres with a diameter between 500 micrometers and 710 micrometers (a maximum diameter of about 786 micrometers) in between 0.5 mL and 1.0 mL saline was placed in a 3 mL syringe and introduced through the catheter to a dish containing saline on a microscope stage. The microspheres were formed from methacrylamide functionalized whey protein. FIGS. 19 and 20 are micrographs of some of the microspheres after introduction through the Boston Scientific Fathom™ 16 catheter. Microsphere compression was up to 24.7%, and, of the visualized microspheres, several microspheres had mild chipping or fracturing, and two microsphere fragments were observed.

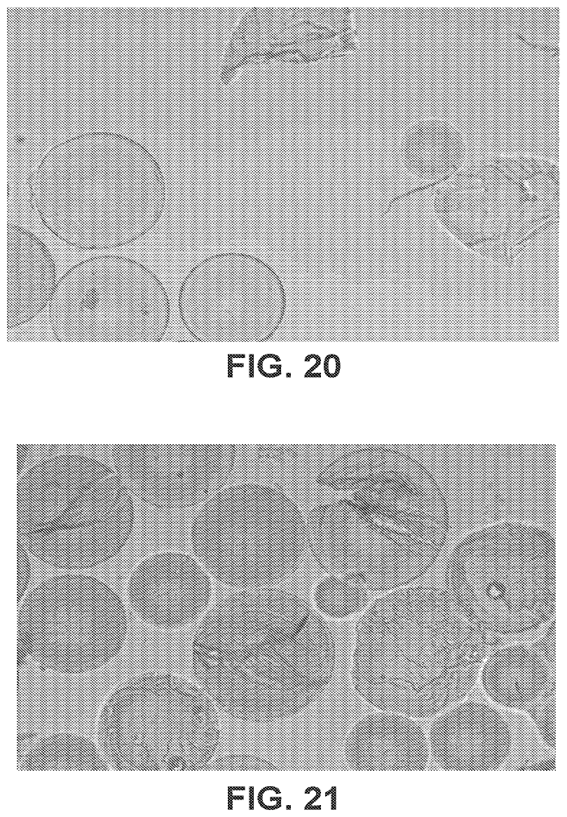

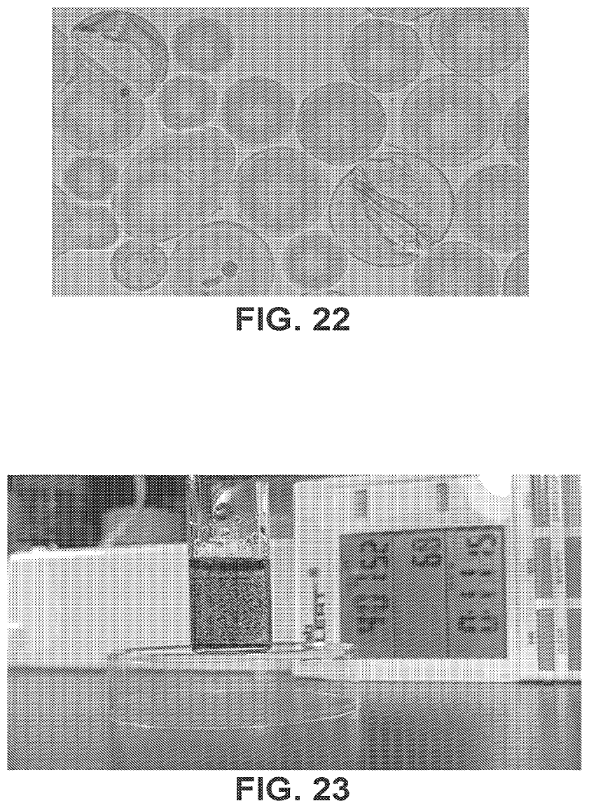

A Concentric Medical Trevo catheter was filled with saline. About 0.2 mL (settled) 20 wt. % microspheres with a diameter between 500 micrometers and 710 micrometers in between 0.5 mL and 1.0 mL saline was placed in a 3 mL syringe and introduced through the catheter to a dish containing saline on a microscope stage. The microspheres were formed from methacrylamide functionalized whey protein. FIGS. 21 and 22 are micrographs of some of the microspheres after introduction through the Concentric Medical Trevo catheter. Microsphere compression was up to 39.1%. During introduction, the catheter clogged several times, but additional pressure cleared the clog each time. Between about 20% and about 30% of the microspheres were damaged with surface or deeper fractures, and a few microspheres fractures into separate pieces. From these the fracture limit is between about 25% and about 39%. At higher compressions, the 20% microspheres had less damage than the 16% microspheres.

### Example 6: Suspension Testing

Microspheres were formed from methacrylamide functionalized whey protein were suspended in a mixture of 50% saline and 50% contrast media. As shown in FIG. 23, the microspheres maintained suspension.

### Example 7: Doxorubicin Loading

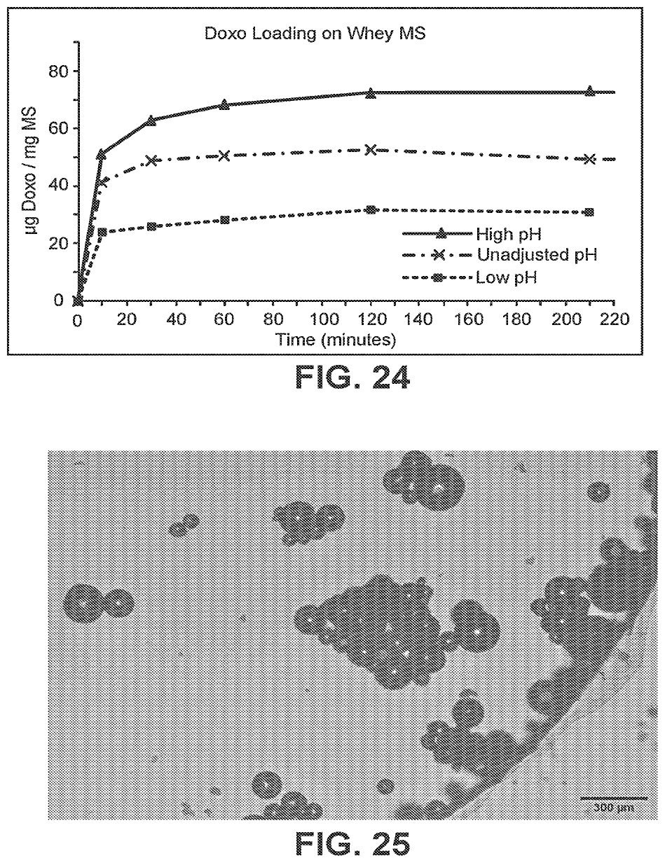

FIG. 24 is a plot of doxorubicin loading on microspheres formed from methacrylamide functionalized whey protein at various pH levels (about 3 for Low pH, about 7 for Unadjusted pH, and about 11 for High pH). As shown in FIG. 24, a higher pH led to a higher doxorubicin loading.

### Example 8: Microsphere Drying and Rehydration

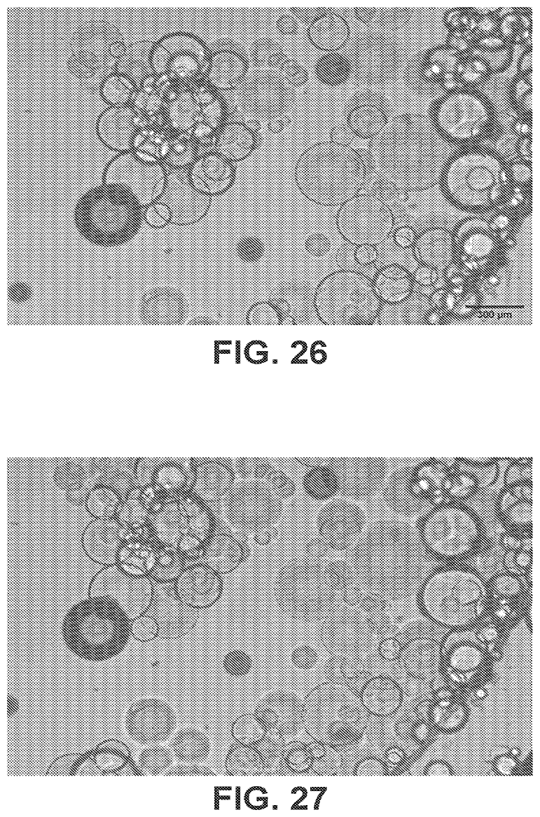

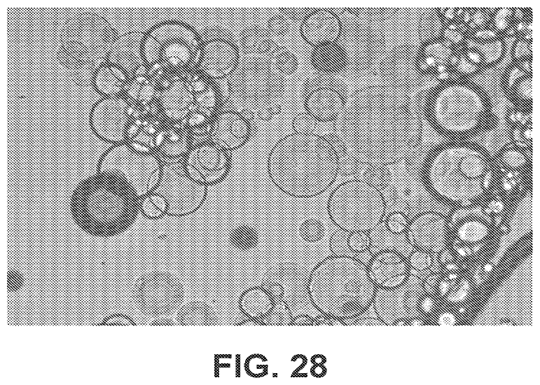

FIGS. 25-28 are micrographs illustrating dried microspheres formed from methacrylamide functionalized whey protein (FIGS. 25) and 2,3, and 40 minutes after rehydration (FIGS. 26-28). To dry the microspheres, a settled volume of approximately 67 μL of microspheres with greater than 106 μm diameter was placed in a 5 mL vial and the excess saline was removed. About 500 μL of 200 proof ethanol was introduced, and the microspheres were allowed to soak for approximately 20 minutes. Excess liquid was removed and the microspheres were allowed to dry for 5 days in the open tube, while air was flowing through a hood. Rehydration was accomplished by adding saline to the dried microspheres.

Various examples have been described. These and other examples are within the scope of the following claims.

1. An embolic material comprising:
a particle comprising a methacrylamide functionalized protein.

2. The embolic material of claim 1, wherein the methacrylamide functionalized protein comprises at least one of a methacrylamide functionalized lysine amino acid group, a methacrylamide functionalized asparagine amino acid group, or a methacrylamide functionalized arginine amino acid group.

3. The embolic material of claim 1, wherein the alkene functionalized biopolymer comprises methacrylamide functionalized whey protein or methacrylamide functionalized soy protein.

4. The embolic material of claim 1, wherein the particle comprises a diameter between about 50 micrometers and about 2000 micrometers.

5. The embolic material of claim 1, wherein the particle comprises at least one of a microsphere or a microcylinder.

6. The embolic material of claim 1, wherein the microsphere further comprises a therapeutic agent.

7. The embolic material of claim 6, wherein the therapeutic agent comprises doxorubicin.

8. The embolic material of claim 1, wherein the microsphere is substantially free of a small molecule crosslinking agent.

9. The embolic material of claim 1, wherein the microsphere is compressible.

10. The embolic material of claim 1, further comprising a second molecule covalently bound to the alkene functionalized biopolymer.

11. The embolic material of claim 10, wherein the second molecule comprises a difunctional molecule.

12. The embolic material of claim 11, wherein the second molecule comprises a diacrylate.

13. An embolization suspension comprising:
a solvent; and
an embolic material comprising a particle comprising a methacrylamide functionalized protein.

14. The embolization suspension of claim 13, wherein the solvent comprises a mixture of contrast medium and saline.

15. The embolization suspension of claim 14, wherein the mixture comprises about 40 vol. % contrast medium and about 60 vol. % saline.

16. A method of forming an embolic material comprising:
introducing a methacrylamide functional group containing a carbon-carbon double bond on a protein to form a methacrylamide functionalized protein; and
crosslinking the methacrylamide functionalized protein to form a plurality of embolic particles.

17. The method of claim 16, wherein the protein comprises at least one of a lysine amino acid group, an asparagine amino acid group, or a arginine amino acid group.

18. The method of claim 16, wherein the protein comprises whey protein or soy protein.

19. The method of claim 16, wherein crosslinking the methacrylamide functionalized protein comprises emulsion polymerizing the methacrylamide functionalized protein to form the plurality of embolic particles.

20. The method of claim 16, wherein crosslinking the methacrylamide functionalized protein comprises free radical initiated polymerizing the methacrylamide functionalized protein to form the plurality of embolic particles.
